(19) United States
(12) Patent Application Publication
(10) Pub. No.: US 2008/0121591 A1
(43) Pub. Date: May 29, 2008

(54) DEVICE FOR NUCLEIC ACID PREPARATION

(75) Inventors: Ivor T. Knight, Arlington, VA (US); Michele R. Stone, Rockville, MD (US); Gregory A. Dale, Gaithersburg, MD (US)

Correspondence Address: ROTHWELL, FIGG, ERNST & MANBECK, P.C., 1425 K STREET, N.W., SUITE 800, WASHINGTON, DC, 20005, US

(73) Assignee: CANON U.S. LIFE SCIENCES, INC., Rockville, MD (US)

(21) Appl. No.: 11/946,503

(22) Filed: Nov. 28, 2007

Related U.S. Application Data
(60) Provisional application No. 60/867,693, filed on Nov. 29, 2006.

(51) Int. Cl. B01D 43/00; B01J 19/00; C12M 1/33

(52) U.S. Cl. 210/748; 210/767; 422/255; 435/306.1

(57) ABSTRACT

A device for nucleic acid preparation includes an outer housing and an inner housing carried within the outer housing and rotatable with respect to the outer housing. The inner housing defines an inner chamber and includes inlet and outlet ports and a nucleic acid extraction substrate within the inner chamber. The outer housing includes a sample inlet port, wash fluid inlet and outlet ports, and elute fluid inlet and outlet ports, and the ports of the inner housing can be selectively aligned with ports of the outer housing by rotating the inner housing with respect to the outer housing. By aligning one or both ports of the inner housing with the appropriate ports of the outer housing, sample material can be introduced into the chamber and wash and elution fluids can be passed through the chamber. Nucleic acid from the sample introduced into the chamber is captured on the extraction substrate and is released from the extraction substrate by sonication prior to being eluted out of the chamber.

This application claims the benefit of Provisional Patent Application Ser. No. 60/867,693, filed on Nov. 29, 2006, which is incorporated herein by reference.

### BACKGROUND OF THE INVENTION

1. Field Of The Invention

The present invention relates to devices in which sample preparation procedures are performed, and, more specifically, sample preparation devices in which nucleic acid is extracted from biological samples.

2. Discussion of Related Art

Purified nucleic acids (DNA and RNA) from tissue samples such as human blood, serum, or saliva can be prepared by first lysing cells by chemicals (chaotropic salts, detergents, and/or strong base) or physical energy such as sonication, and then purifying the nucleic acids from other cellular components in the cell lysate. Historically, organic extraction (e.g., phenol:chloroform) followed by ethanol precipitation was used to purify the nucleic acids. This extraction method has been largely replaced by solid phase extraction methods that do not use phenol or chloroform. Various matrices have been used for solid phase extraction, most commonly silica particles. Nucleic acids bind to silica in the presence of high concentrations of chaotropic salts (Chen and Thomas, 1980; Marko et al. 1982; Boom et al. 1990). These salts are then removed with an alcohol-based wash and the DNA eluted in a low ionic strength solution such as TE buffer or water. The binding of DNA to silica seems to be driven by dehydration and hydrogen bond formation, which competes against weak electrostatic repulsion (Melzak et al. 1996). Hence, a high concentration of salt will help drive DNA adsorption onto silica, and a low concentration will release the DNA.

Recently, new solid phase materials for DNA purification have been developed which take advantage of the negatively charged backbone of DNA to a positively charged solid substrate (under specific pH conditions), and eluting the DNA using a change in solvent pH, such as, for example, Invitrogen's charge switch technology (See United States Patent Application Publication No. 2006-0024712 A1). Whatman has an alternate technology (FTA paper, Whatman plc, Kent, UK) that utilizes a cellulose based solid substrate impregnated with a lysis material which lyses cells, inactivates proteins, and captures DNA in the cellulose fibers, where it is retained for use in downstream applications (See U.S. Pat. No. 6,322,983 B1).

Typically, the above nucleic acid extraction methods are carried out in tubes or microwell plates, either by manual process or by automated processes. Pipetting devices are used to transfer the various liquids utilized in the process and centrifugation can be used to collect the solid phase materials. Alternatively, magnetized solid phase materials can be used, enabling the collection of the solid phase material by use of a magnet.

Existing devices for automated solid phase extraction of nucleic acids are robotic pipetting or vacuum systems utilizing open tubes which are easily contaminated when processing multiple samples. Thus, there is a need for a device that integrates sample collection, storage, and processing within the same container to prevent contamination, carryover, and errors in sample handling throughout the multiple step process of nucleic acid purification.

### SUMMARY OF THE INVENTION

In one aspect, the present invention relates to a sample preparation device that integrates sample collection, storage, and nucleic acid extraction. The sample preparation device is designed to integrate with downstream processes for nucleic acid analysis, such as polymerase chain reaction (“PCR”).

The device includes an inner housing with a chamber containing a solid nucleic acid extraction substrate having inlet and outlet ports and an outer housing with a sample port for delivering a liquid tissue sample, optionally a hypodermic needle protruding from the sample port attached to a capillary from the needle to the interior of the chamber, a set of two ports for flowing wash buffer through the chamber containing the solid substrate, and a set of two ports for eluting the nucleic acid sample from the extraction substrate.

In a second aspect, the present invention provides a method of using the device that includes aligning one of the chamber ports of the inner housing with the sample input port and then introducing a sample material into the inner chamber under conditions that will allow nucleic acid within the sample to bind to the extraction substrate. The chamber inner ports are then aligned with wash ports and wash fluid is introduced into and removed from the chamber through the wash ports. Ultrasonic energy is then applied to release nucleic acid from the extraction substrate. The chamber inner ports are then aligned with the elute ports and elution fluid is introduced into and removed from the chamber through the elute ports.

Thus, the device is a closed system for sample preparation. After sample is introduced, all processes occur within the cartridge until the purified nucleic acid extract is expelled. The advantage is that there is less chance of crossover, contamination, and errors in sample identification.

In a third aspect, a genomic isolation chamber is provided which comprises a housing, a chamber within the housing, a first set of channels, wherein the first set of channels is configured to deliver a first medium to the chamber and extract a second medium from the chamber. The housing also includes a second set of channels that is configured to deliver a third medium to the chamber and extract a fourth medium from the chamber. An input channel is also provided that is configured to deliver a sample to the chamber. The genomic isolation chamber further comprises an adsorption substrate that is configured to adsorb at least a portion of nucleic acid in the sample. The genomic isolation chamber is configured so that a combination of flowing the first, second, third, and fourth mediums through or from the chamber isolates the adsorbed nucleic acid in the fourth medium. The nucleic acid is released from the adsorption substrate by using either sonication from a sonic emitter inserted through the input channel or by noncontact sonication.

Other aspects of the present invention, including the methods of operation and the function and interrelation of the elements of structure, will become more apparent upon consideration of the following description and the appended claims, with reference to the accompanying drawings, all of which form a part of this disclosure, wherein like reference numerals designate corresponding parts in the various figures.

### BRIEF DESCRIPTION OF THE DRAWINGS

FIG. 1 is perspective view of a device for nucleic acid preparation embodying aspects of the present invention;

FIG. 2 is a cross-sectional view of the device along the line 2-2 in FIG. 1;

FIG. 3 is a cross-sectional view of the device along the line 3-3 in FIG. 1; and

FIG. 4 is a flow chart illustrating a nucleic acid preparation procedure embodying aspects of the present invention.

### DETAILED DESCRIPTION OF THE INVENTION

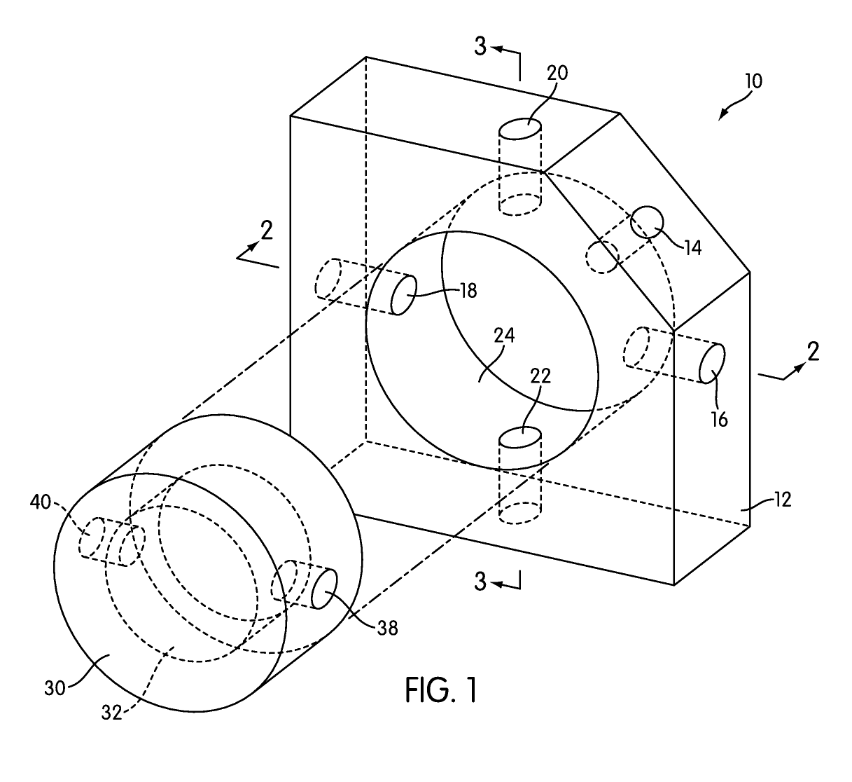

A device for nucleic acid preparation embodying aspects of the present invention is shown in FIGS. 1-3 and includes a cartridge designated by reference number 10. Cartridge 10 includes an outer housing 12 and an inner housing 30. Inner housing 30 is circular and fits within a circular opening 24 formed in the outer housing 12. The inner housing 30 is mounted so as to be rotatable about its axis within the opening 24 of the outer housing 12.

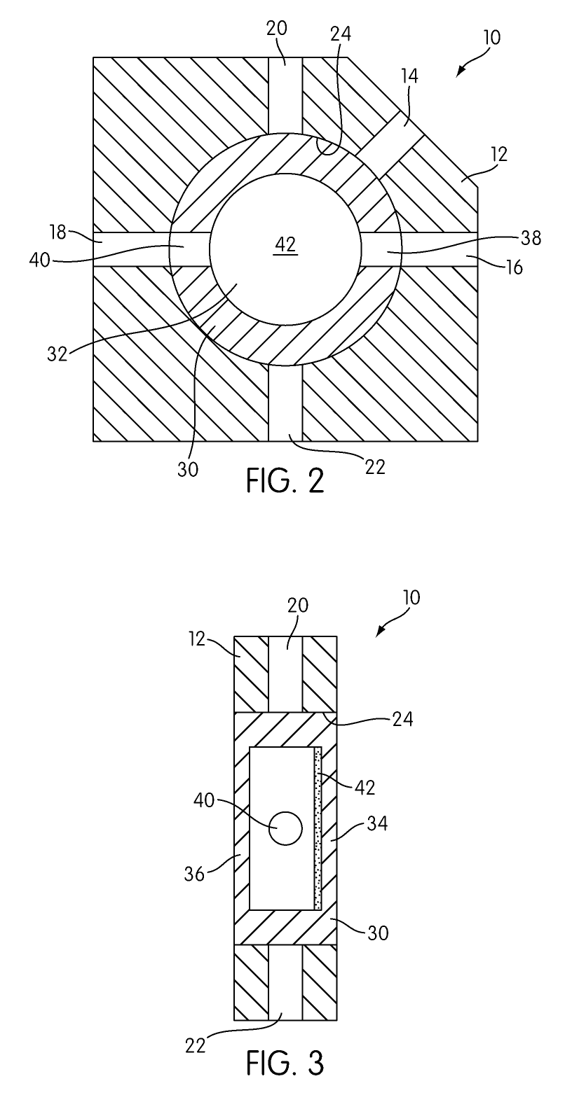

Inner housing 30 defines an inner chamber 32 surrounded by the outer periphery of the inner housing 30 and radial sidewalls 34 and 36. The inner chamber 32 is preferably circular with first and second ports 38, 40 formed preferably at 180 degrees from each other and communicating with the inner chamber 32. One of the ports 38, 40 will function as an inlet port for introducing materials into the chamber 32, and the other port will function as an outlet port. A solid nucleic acid extraction substrate, indicated by reference number 42, is disposed within the inner chamber 32. In one non-limiting embodiment, the nucleic acid extraction device 42 is disposed against the sidewall 34 of the inner housing 30, as illustrated in FIG. 2.

The outer housing 12 includes a sample input port 14, first and second wash ports 16, 18 formed preferably at 180 degrees from each other, and first and second elute ports 20, 22 formed preferably at 180 degrees from each other. Outer housing 12 may include a hypodermic needle (not shown) protruding from the sample input port 14 attached to a capillary from the needle to the interior of the inner chamber 32. The inner housing 30 fits inside the outer housing 12 and is able to rotate so as to selectively align the first and second inner ports 38, 40 with ports formed in the outer housing. In FIG. 2, inner housing 30 is shown positioned so that first and second inner ports 38, 40 are aligned with first and second wash ports 16, 18. Alternatively, the inner housing 30 can be held fixed and the outer housing 12 rotated (or both inner housing 30 and outer housing 12 can be rotated) to effect selective alignment of inner and outer housing ports.

It will be appreciated that ports 16 and 18, 20 and 22, and 38 and 40 need not necessarily be 180 degrees from each other. It is merely necessary that ports 16 and 18 and ports 20 and 22 of the outer housing 12 be angularly spaced by the same amount as ports 38 and 40 of the inner housing 30, so that the ports of the inner housing 30 can be aligned with the ports of the outer housing 12. Also, it is not necessary for the implementation of the invention that first and second wash ports 16, 18 and the first and second elute ports 20, 22 be aligned along mutually orthogonal directions as shown in FIG. 2.

The sample input port 14 can be used to deliver a sample material to the inner chamber 32 when one of the first and second ports 38, 40 of the inner housing 30 is aligned with the sample input port 14. The first and second wash ports 16, 18 can be used to flow a wash fluid through the inner chamber 32 when the first and second ports 38, 40 of the inner housing 30 are aligned with the first and second wash ports 16, 18. Finally, the first and second elute ports 20, 22 can be used to flow an elution fluid through the inner chamber 32 when the first and second inner ports 38, 40 of the inner housing 30 are aligned with the first and second elute ports 20, 22.

Cartridge 10 is preferably disposable and may be incorporated within a closed system or instrument, thereby decreasing the likelihood of carryover, errors, or contamination which can occur during the multi-step process of nucleic acid purification. Thus, one of the first and second wash ports 16, 18 may be connected to a source (e.g., a pump, chamber, compartment, or container) of a wash fluid, and the other wash port may be connected to a waste chamber. Similarly, one of the first and second elute ports 20, 22 may be connected to a source (e.g., a pump, chamber, compartment or container) of an elution fluid, and the other elute port may be connected to a compartment for storing the nucleic acid or to other downstream processing. Alternatively, compartments for storage of wash fluid, elution fluid, wastes, and extracted nucleic acid can be formed in the cartridge itself, for example in the outer housing. Rotation of the inner housing 30 (or, alternatively, of the outer housing 12) can be effected by any suitable means, such as mechanical, electromechanical, pneumatic, magnetic, piezoelectric, or other actuator means. Furthermore, the outer shape of the outer housing 12 shown in FIGS. 1-3 is for illustration only; the outer housing 12 may have any outer shape so as to conform to a system or instrument in which it is incorporated.

As mentioned, the inner chamber 32 contains a nucleic acid extraction substrate 42 for capturing the components of the sample to be lysed. Suitable substrates generally include filters, beads, fibers, membranes, glass wool, filter paper, polymers, and gels. The substrate may capture the desired sample components through physical retention, e.g., adsorption, size exclusion, through affinity retention, or through chemical interaction. Suitable filter materials include glass, fiberglass, nylon, nylon derivatives, cellulose, cellulose derivatives, and other polymers. In an alternative embodiment, the substrate comprises polystyrene, silica, agarose, cellulose, or acrylamide beads. In the presently preferred embodiment, the substrate comprises a membrane or filter formed from FTA paper (Whatman plc, Kent, UK). FTA paper, as described in U.S. Pat. No. 6,322,983, the disclosure of which is incorporated by reference, utilizes a cellulose based solid substrate impregnated with a lysis material which lyses cells, inactivates proteins, and captures nucleic acid in the cellulose fibers.

In one embodiment, the nucleic acid preparation procedure performed in accordance with the present invention includes the use of suitable energy applied to the nucleic acid extraction substrate 42 to release the captured nucleic acid from the nucleic acid extraction substrate 42. For example, any mechanical energy suitable to dislodge the captured nucleic acid from the nucleic acid extraction substrate 42 can be used, and such energy can be generated by any suitable energy-emitting device. Preferably, the mechanical energy comprises pressure waves emitted by a pressure wave-emitting device. The pressure wave emitting device may comprise an acoustic energy emitting device. The acoustic energy emitting device produces acoustic energy that is used to release genetic material from a solid support. Any device that generates sound waves can be used as a source of acoustic energy. Such devices include, but are not limited to, ultrasonic transducers, piezoelectric transducers, magnorestrictive transducers, and electrostatic transducers. Suitable devices are well known in the art and include such commercially available devices as the Sonicator 4000 (Misonix, Inc., Farmingdale, N.Y., USA), Microson® Sonicator Microprobe or Micro Cup Horn (Kimble/Kontes, Vineland, N.J., USA) and Covaris™ Adaptive Focused Acoustics (Nexus Biosystems, Poway, Calif., USA). Other suitable devices are described in U.S. Pat. Nos. 6,881,541 and 6,878,540 and in U.S. Patent Application Publication No. 2007/0170812.

Thus, according to one embodiment, the device includes, or is used in conjunction with, an ultrasonic transducer, such as an ultrasonic horn for non-contact sonication. In another aspect, this embodiment includes, or is used in connection with, an ultrasonic probe for contact sonication that is coupled to the cartridge 10 for transferring ultrasonic energy to the components captured on the substrate 42. Contact sonication is performed by introducing a probe sonicator (ultrasonic probe) into the inner chamber 32 through the sample input port 14 and one of the first or second inner ports 38, 40 aligned with port 14. Alternatively, non-contact sonication is performed by placing a non-contacting sonicator (e.g., an ultrasonic horn) adjacent the side wall 34 of the inner housing 30 that is in close proximity to the substrate 42. To aid in the transfer of ultrasonic energy to the sample components if non-contact sonication is to be employed with the cartridge 10, it is preferred that side wall 34 be a relatively thin film or membrane, preferably having a thickness in the range of 0.01 to 0.5 mm, and more preferably have a thickness of about 0.05 mm.

The cartridge 12 may be fabricated of any suitable polymer and may be fabricated using conventional techniques. In particular, the inner housing 30 and the outer housing 12 preferably comprise molded plastic. Examples of suitable plastic materials for the inner housing 30 and the outer housing 12 (including side wall 34 if it comprises a thin film or membrane for non-contact sonication) include, e.g., polycarbonate, polystyrene, polypropylene, polyethylene, acrylic, and commercial polymers. Substrate 42 may optionally be heat sealed within the inner chamber 32 of the inner housing 30.

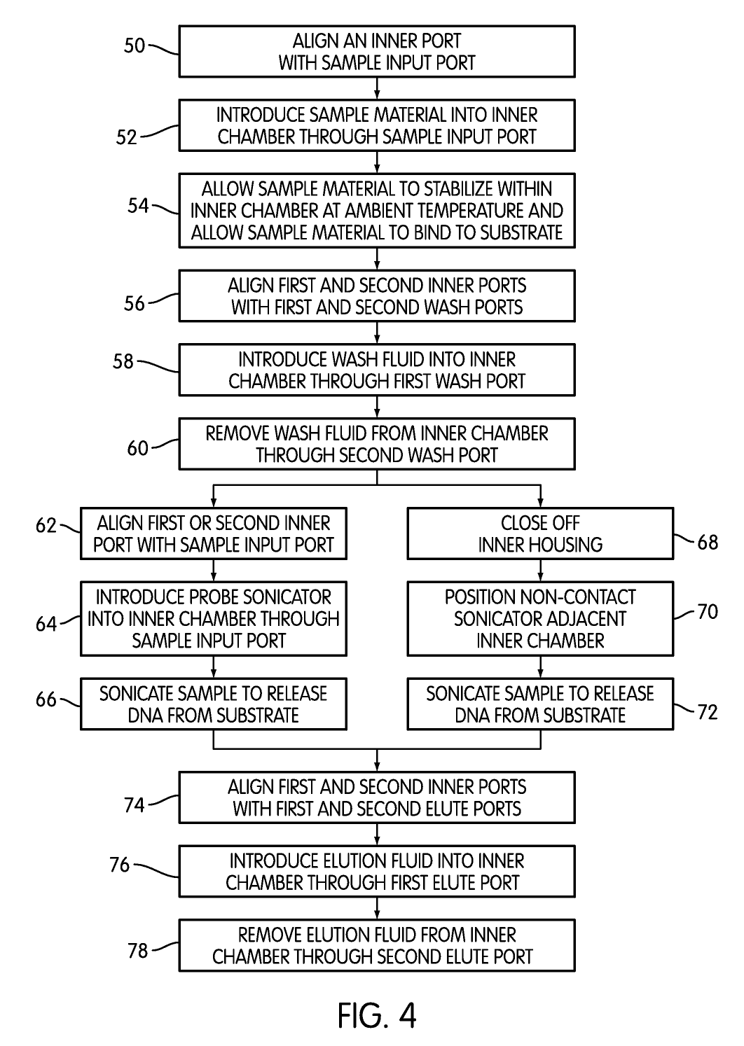

FIG. 4 is a flow chart illustrating a process for nucleic acid extraction in accordance with the present invention using the cartridge 10. In Step 50, inner housing 30 is moved (rotated) to align the first or the second inner port 38, 40 of the inner housing 30 with the sample input port 14 of the outer housing 12 to open inner chamber 32. In Step 52, sample material (e.g., whole blood, saliva, cerebro spinal fluid, amniotic fluid, serum, urine, fluid from a vaginal or buccal swab, or tissue sample) is introduced into inner chamber 32 through sample input port 14 and the first or second inner port 38, 40 with which the sample input port 14 is aligned. In Step 54, the sample material temperature is allowed stabilized within inner chamber 32 at ambient temperature, and the sample material is allowed to bind to substrate 42.

Next, in Step 56, inner housing 30 is moved (rotated) to align the first and second inner ports 38, 40 of the inner housing 30 with first and second wash ports 16, 18, respectively, of the outer housing 12. In Step 58, wash fluid (e.g., water) is introduced from a wash fluid source (e.g., a pump, compartment, chamber, or container (not shown)) into the inner chamber 32 through first wash port 16 and aligned first inner port 38, and, in Step 60, the wash fluid is removed from inner chamber 32 through second wash port 18 and aligned second inner port 40. The removed wash fluid may be transmitted to a waste chamber or container (not shown). The direction of flow of the wash fluid can be reversed, that is, from second wash port 18 to first wash port 16.

In Step 62, inner housing 30 is moved (rotated) to align the first or second inner port 38, 40 with sample input port 14 to open inner chamber 32. Then, in Step 64, a probe sonicator is introduced into inner chamber 32 through sample input port 14 and aligned first or second inner port 38, 40. The probe is introduced into fluid contained within inner chamber 32, and, in Step 66, the sample is sonicated to release nucleic acid from substrate 42 in a procedure referred to as contact sonication.

As an alternative to contact sonication, nucleic acid may be released from substrate 42 by non-contact sonication. A non-contact sonication procedure is performed by first, in Step 68, moving inner housing 30 so the first and second inner port 38, 40 are not aligned with any ports of the outer housing 12, to thereby seal off the inner chamber 32. In Step 70, a non-contact sonicator (e.g., an ultrasonic horn) is positioned adjacent chamber side wall 34, which, to enable such a process, and as described above, is made of a thin membrane material constructed and arranged to enable the transmission of sufficient sonic energy to release nucleic acid from the substrate 42. In Step 72, the sample is sonicated by the non-contact sonicator to release nucleic acid from substrate 42. In a non-limiting example, a frequency of approximately 20 kH can be used for a duration of approximately 15 seconds to 60 seconds to achieve the desired release of at least a portion of nucleic acid. Of course, other sonical frequencies and duration of sonication can be used as well.

Following sonication by the contact or non-contact procedures described above, in Step 74, the inner housing 30 is moved to align the first and second inner ports 38, 40 of the inner housing 30 with first and second elute ports 20, 22, respectively, of the outer housing 12. In Step 76, elution fluid (e.g., an elution fluid that is compatible for downstream processing and/or storage) is introduced from an elution fluid source (e.g., a pump, compartment, chamber, or container (not shown)) into the inner chamber 32 through first elute port 20 and aligned first inner port 38. In Step 78, the elution fluid is removed from inner chamber 32 through second elute port 22 and aligned second inner port 40, to remove the released nucleic acid from the chamber. The nucleic acid is then transmitted to a compartment for storage or transmitted downstream for further processing. In another embodiment, the direction of flow of the elution fluid can be reversed, that is, from second elute port 22 to first elute port 20.

In another aspect of the invention, a genomic isolation chamber is disclosed which comprises a housing 12, a chamber 42 within the housing, a first set of channels 16, 18, wherein the first set of channels is configured to deliver a first medium (e.g. a wash medium) to the chamber and extract a second medium (e.g. wash medium plus contaminants) from the chamber. See FIGS. 1-3. The housing also includes a second set of channels 20, 22 that is configured to deliver a third medium (e.g. an elution medium) to the chamber and extract a fourth medium (elution medium plus nucleic acid) from the chamber. An input channel 14 is also provided that is configured to deliver a sample to the chamber. The genomic isolation chamber further comprises an adsorption substrate 42 that is configured to adsorb at least a portion of nucleic acid in the sample. The genomic isolation chamber is configured so that a combination of flowing the first, second, third, and fourth mediums through or from the chamber isolates the adsorbed nucleic acid in the fourth medium. The nucleic acid is released from the adsorption substrate by using either sonication from a sonic emitter inserted through the input channel or by noncontact sonication.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A device for nucleic acid preparation comprising:
a first part defining a chamber therein and having first and second ports communicating with said chamber;
a nucleic acid extraction substrate contained within said chamber; and
a second part having a sample input port, first and second wash ports, and first and second elute ports,
wherein said first and second parts are configured in operative relation with each other and are moveable with respect to each other so that (a) one of said first and second ports of said first part can be selectively aligned with said sample input port to permit a sample material to be introduced to said chamber through said sample input channel, (b) said first and second ports of said first part can be selectively aligned with said first and second wash ports to permit a wash medium to be introduced to said chamber through one of said first and second wash ports and removed from said chamber through the other of said first and second wash ports, or (3) said first and second channels of said first part can be selectively aligned with said first and second elute ports to permit an elution medium to be introduced to said chamber through one of said first and second elute ports and removed from said chamber through the other of said first and second elute ports.

2. The device of claim 1, wherein the first part and second part are molded from at least one material selected from the group of materials consisting of polycarbonate, polystyrene, polypropylene, polyethylene, and acrylic.

3. The device of claim 1, wherein said first part includes a side wall at least partially enclosing the chamber, and wherein at least a portion of said side wall comprises a thin membrane constructed and arranged to allow transmission of energy through the membrane.

4. The device of claim 3, wherein said membrane is constructed and arranged to allow transmission of ultrasonic energy through the membrane.

5. The device of claim 4, wherein said membrane has a thickness of 0.01-0.5 mm.

6. The device of claim 5, wherein said membrane has a thickness of about 0.05 mm.

7. The device of claim 1, wherein said nucleic acid extraction substrate comprises a cellulose-based solid substrate impregnated with lysis material which lyses cells and captures nucleic acid in the cellulose fibers.

8. The device of claim 1, wherein said first part is circular in shape, and wherein said second part includes a circular opening for receiving said first part therein, and wherein said first part is configured to be rotatable within the circular opening of said second part.

9. The device of claim 8, wherein the first and second ports of said first part are disposed on opposite sides of said first part and are spaced from each other by about 180°.

10. The device of claim 9, wherein the first and second wash ports of said second part are disposed on opposite sides of the circular opening and are spaced from each other by about 180°, and the first and second elute ports of said second part are disposed on opposite sides of the circular opening and are spaced from each other by about 180°.

11. The device of claim 10, wherein the first and second wash ports and the first and second elute ports of said second part are aligned along mutually orthogonal directions.

12. A method for extracting nucleic acid from a sample within a cartridge comprising a first part defining a chamber therein and having first and second ports communicating with said chamber and a nucleic acid extraction substrate contained within said chamber and a second part having a sample input port, first and second wash ports, and first and second elute ports, wherein said first and second parts are configured in operative relation with each other and are moveable with respect to each other, said method comprising the steps of:
moving the first and second parts with respect to each other to align the first or second port of the first part with the sample input port of the second part;
introducing sample material into the inner chamber through the sample input port and the aligned first or second inner port under conditions that will allow nucleic acid within the sample material to bind to the nucleic acid extraction substrate;
moving the first and second parts with respect to each other to align the first and second ports of the first part with the first and second wash ports, respectively, of the second part;
introducing wash fluid into the inner chamber through the first wash port and the aligned first port;
removing wash fluid from the inner chamber through the second wash port and the aligned second inner port;
applying energy to release nucleic acid from the substrate;
moving the first and second parts with respect to each other to align the first and second ports of the first part with the first and second elute ports, respectively, of the second part;
introducing an elute medium into the inner chamber through the first elute port and the aligned first port; and
removing elute medium from the inner chamber through the second elute port and the aligned second inner port.

13. The method of claim 12, wherein said step of applying energy is applying ultrasonic energy.

14. The method of claim 13, wherein applying ultrasonic energy comprises:
moving the first and second parts with respect to each other to align the first or second port of the first part with the sample input port of the second part;
introducing a probe sonicator into the inner chamber through sample input port and aligned first or second port so that the probe sonicator is in contact with the contents of the inner chamber; and
applying ultrasonic energy from the probe sonicator to the contents of the inner chamber.

15. The method of claim 13, wherein applying ultrasonic energy comprises:
moving the first and second parts with respect to each other so that neither the first nor second port of the first part is aligned with any port of the second part;
positioning a sonicator exterior to the inner chamber adjacent an outer wall of the inner chamber; and
applying ultrasonic energy to the outer wall of the inner chamber in such a manner that sufficient ultrasonic energy is transmitted through the outer wall to release nucleic acid from the substrate.

16. The method of claim 12, wherein the sample material comprises at least one of whole blood, saliva, spinal fluid, amniotic fluid, serum, urine, or fluid from a vaginal or buccal swab.

17. The method of claim 12, wherein the wash fluid comprises water.

18. The method of claim 12, wherein the nucleic acid extraction substrate comprises a cellulose-based solid substrate impregnated with lysis material which lyses cells and captures nucleic acid in the cellulose fibers.

19. The method of claim 12, wherein the ports of the first part are aligned with ports of the second part by rotating the first part with respect to the second part.

20. A genomic isolation chamber comprising:
a housing;
a chamber within the housing;
a first set of channels, wherein the first set of channels is configured to deliver a first medium to the chamber and extract a second medium from the chamber;
a second set of channels, wherein the second set of channels is configured to deliver a third medium to the chamber and extract a fourth medium from the chamber;
an input channel, wherein the input channel is configured to deliver a sample to the chamber; and
an adsorption substrate, wherein the adsorption substrate is configured to adsorb at least a portion of nucleic acid in the sample, and wherein the genomic isolation chamber is configured so that a combination of flowing the first, second, third, and fourth mediums through or from the chamber isolates the adsorbed nucleic acid in the fourth medium.

21. The genomic isolation chamber according to claim 20, wherein the nucleic acid is released from the adsorption substrate by using either sonication from a sonic emitter inserted through the input channel or by noncontact sonication.
